(19) United States
(12) Patent Application Publication
(10) Pub. No.: US 2006/0200250 A1
(43) Pub. Date: Sep. 7, 2006

(54) Biocompatible implant device

(76) Inventor: David N. Ku, Atlanta, GA (US)

Correspondence Address: JONES DAY, 555 SOUTH FLOWER STREET FIFTIETH FLOOR, LOS ANGELES, CA, 90071, US

(21) Appl. No.: 11/073,337

(22) Filed: Mar. 4, 2005

(51) Int. Cl. A61F 2/04

(52) U.S. Cl. 623/23.71

(57) ABSTRACT

A medical implant made of an elastic, solid, biocompatible, and non-hemolytic material having a thickness of less than about 5 mm, a modulus of elasticity between about 10 kPa and about 100 MPa. The implant device has a pore size of less than about 10 microns in order to prevent growth and passage of cells through the implant, while allowing water and nutrient transport across the implant device.

### FIELD OF THE INVENTION

The present invention relates to a tubular medical implant device made of an elastic, solid substance having a thickness of less than about 5 mm, a modulus of elasticity between about 10 kPa and about 100 MPa with a biocompatible, and non-hemolytic surface. The implant device has a pore size of less than about 10 microns in order to prevent growth and passage of cells through the implant wall, while allowing water and nutrient transport across the implant device wall.

### DESCRIPTION OF THE PRIOR ART

Medical devices may be used to reconstruct organs and soft-tissues in the body. An ideal medical device could interact with the cells of the body to provide lasting structural support without causing inflammation or interfering with the normal physiologic functions of nearby organs. It would be useful to have devices that separate one layer of cells from another to maintain tissue planes while allowing for transport of nutrients to the cells. In reality, no such ideal medical devices are commercially available. The need for a cellular barrier remains.

Some medical implants can provide a structural scaffold for the ingrowth of cells. One example might be a biodegradable poly-glycolic acid scaffold that has the shape of a human ear. It has been demonstrated that a fibrotic “ear” can be grown on the back of a mouse using this technique. (D. J. Mooney, A. G. Mikos. Growing New Organs. Scientific American 280, 60-65 (April 1999); Vicanti and Langer, http://www.pbs.org/saf/1107/features/body.htm) A problem with this technique is that the device structure soon degrades into a mush with a very high acidity that may kill cells. Other materials such as polyethyleneterephthalate (Polyester) can be made into meshes to cover hernia openings. A common problem with such devices is a large inflammatory response that can cause massive amounts of local tissue reaction, fibrosis, or hyperplasia.

Some implant devices have a pore size that allow the passage of cells through the implant wall and further allows for undesirable cellular growth within implant cavities. Indeed, many implant are purposely designed with a large pore size to promote tissue in-growth to anchor the implant.

Numerous references generally describe the process of freezing and thawing PVA to create a hydrogel: Chu et al., Poly(vinyl alcohol) Cryogel: An Ideal Phantom Material for MR Studies of Arterial Elasticity, Magnetic Resonance in Medicine, v. 37, pp. 314-319 (1997); Stauffer et al., Poly(vinyl alcohol) hydrogels prepared by freezing-thawing cyclic processing, Polymer, v. 33, pp. 3932-3936 (1992); Lozinsky et al., Study of Cryostructurization of polymer systems, Colloid & Polymer Science, v. 264, pp. 19-24 (1986); Watase and Nishinari, Thermal and rheological properties of poly(vinyl alcohol) hydrogels prepared by repeated cycles of freezing and thawing, Makromol. Chem., v. 189, pp. 871-880 (1988). The disclosure from these references is hereby incorporated by reference.

Another such reference is U.S. Pat. No. 4,734,097, issued to Tanabe et al. on Mar. 29, 1988 (“Tanabe”). Tanabe proposes the construct of a molded hydrogel obtained by pouring an aqueous solution containing not less than 6% by weight of a polyvinyl alcohol which has a degree of hydrolysis not less than 97 mole percent and an average polymerization degree of not less than 1,100 into a desired shape of a vessel or mold, freeze molding an aqueous solution in a temperature lower than minus 5° C., then partially dehydrating the resulting molded product without thawing it up to a percentage of dehydration not less than 5 weight percent, and if required, immersing the partially hydrated molded part into water to attain a water content thereof in the range of 45 to 95 weight percent.

The disadvantage to Tanabe et al. is that it necessarily requires a step of dehydration in preparing the PVA hydrogel. There are several disadvantages associated with the dehydration step. First, the dehydration step adds additional time and capital expense associated with machinery which must accomplish the dehydration step. Additionally, dehydration may denature bioagents included in the hydrogel.

Hyon et al., U.S. Pat. No. 4,663,358 is directed to producing PVA hydrogels having a high tensile strength and water content. However, this patent is not directed to hydrating the PVA with water alone, but rather uses a mixture of water and an organic solvent such as dimethyl sulfoxide (DMSO). DMSO is recognized as an initiator of carcinogenicity. Residual amounts of organic solvents in the resultant PVA hydrogel render such products undesirable for biomedical applications, particularly where the hydrogel is to be used for long term implants within the body.

With the foregoing disadvantages of the prior art in mind, it is an object of the present invention to provide an implant device that prevents growth and passage of cells through the implant, while allowing water and nutrient transport across the implant device.

Other objects, features and advantages of the present invention will become apparent upon reading the following specification.

### SUMMARY OF THE INVENTION

The present invention relates to a medical implant having certain unique properties. The implant is made of an elastic, biocompatible and non-hemolytic substance with the solid portion having a thickness of less than 5 mm, a modulus of elasticity between about 10 kPa and about 100 MPa. The implant has a pore size of less than about 10 microns that prevents growth and passage of cells through the implant wall, while allowing water and other nutrients to be transported through the implant wall.

The implant may be formed in a tubular shape about a cylindrical mandrel. The implant may be made from a mixture of polyvinyl alcohol and water and subjected to at least one freeze-thaw cycle.

### BRIEF DESCRIPTION OF THE DRAWINGS

FIG. 1 is a perspective view of the implant device of the claimed invention.

FIG. 2 is a side view of the implant device of the claimed invention.

FIG. 3 is a front view of the implant device of the claimed invention formed in a tubular shape about a mandrel.

### DETAILED DESCRIPTION OF PREFERRED EMBODIMENTS

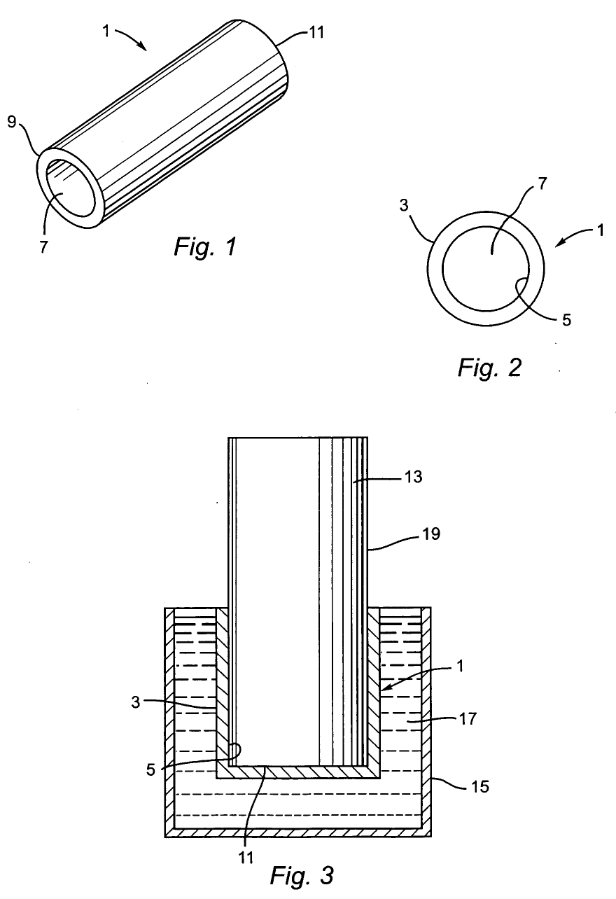

FIG. 1 illustrates an implant device 1. Preferably implant device 1 is made of an elastic, solid biocompatible and non-hemophilic device having a modulus of elasticity between about 10 kPa and about 100 MPa. More preferably, the implant device is of tubular shape. FIG. 2 shows the implant device 1 as viewed from the side. The implant device has an outer surface 3 and an inner surface 5. The thickness of the implant device 1, measured as the distance between the outer surface 3 and the inner surface 5 is uniform and less than about 5 mm. In a preferred embodiment, the implant device 1 has a pore size of less than 10 microns that prevents growth and passage of cells from outer surface 3 to inner surface 5, while allowing water and nutrient transport from outer surface 3 to inner surface 5.

The implant device 1 has an opening 7, preferably generally circular at a first end 9. An opposite end 11 may be open to allow for flow, such as blood flow, through the implant 1, or closed to prevent flow through the implant device 1, when, for example, the implant device is positioned about a ruptured blood vessel or “bleeder” in order to prevent bleeding. Alternatively, an implant device 1 having a closed opposite end 11 may be employed as a fallopian tube barrier.

FIG. 3 illustrates the formation of the implant device 1. A solid, preferably circular, mandrel 13 may be placed in a container 15 holding a synthetic organic polymer 17. Preferably, the synthetic organic polymer is a mixture of polyvinyl alcohol (PVA) and water. A method of forming a polyvinyl alcohol construct is more particularly described in U.S. Pat. Nos. 6,231,605 and 5,981,826, hereby incorporated by reference.

When the mandrel 13 is inserted into the synthetic organic polymer 17, the viscosity of the liquid synthetic organic polymer 17 causes the synthetic organic polymer 17 to adhere to the outer surface 19 of the mandrel 13. The mandrel 13 may be spun in order to allow the synthetic organic polymer 17 to coat the mandrel to a uniform thickness, preferably less than about 5 mm. The mandrel is removed from the liquid polymer and the device is then sequentially frozen and thawed, at least once.

As shown in FIG. 3, the opposite end 11 of implant device 1 is closed. Alternatively, when the implant device 1 and mandrel 13 are removed from the container 17, and the implant device 1 is removed from the mandrel 13, the opposite end 11 of the implant device 1 may be cut to produce an opening similar to opening 7 at first end 9. The final device may allow cellular in-growth and fluids to pass from one end while preventing cellular penetration along the walls of the device.

Alternatively, devices may be made with a cast-mold process that is common in manufacturing. These mold surfaces may include surface textures to create devices with specific pore sizes and cellular adhesion properties. Further, other materials such as cross-linked polymers such as polyurethanes, poly-caprolactones, collagen, elastin or irradiated PVA devices are anticipated as possible manufacturing substances to make the medical devices describe here.

Preferably, the implant device 1 has an ultimate stretch greater than 200 percent in order to allow the implant device 1 to be stretched over a support implant structure. Preferably, the device will not melt under body temperatures and thus will have a melting point exceeding 42 degrees Celcius, nor should the device dissolve in saline. Further, the implant device is preferably both radiolucent and clear, and is hydrophilic and may absorb water without degrading in vivo. The implant device 1 is preferably of a diameter that allows for delivery by catheter. More preferably, the implant device 1 may be sterilized such that it may be used in the cardiovascular system and in contact with blood. Further, the device may include a mechanism for anchoring the device in the body such as with a suture or metallic appendage. The device may act as a physical exclusion barrier or sieve to allow cells of a certain size to pass through the device in certain directions, but not in other directions, termed penetration anisotropy. Further, the device may be made with hydrophilic molecules such as sugars, proteins, proteoglycans, cytokines, or cytostatic agents.

The devices reduced to practice by this method have been tested to demonstrate the claimed properties. The devices are solid yet allow for water transport by diffusion and convection. When immersed in saline, the devices are stable and do not dissolve or melt up to 45 degrees Celcius. Oxygen nutrients and other water soluble molecules diffuse through the walls of the device. The device made as described as a preferred manufacturing method from aqueous PVA is biocompatible, being tested in accordance with ISO 10993, “Biological Evaluation of Medical Devices,” and the guidance document released by FDA in 1995, blue book memorandum #G95-1, “Use of International Standard ISO 10993, ‘Biological Evaluation of Medical Devices'—Part 1: Evaluation and Testing. Similarly, hemolysis tests also demonstrate the preferred device material of aqueous PVA is non-hemolytic. The preferred devices constructs have an elastic modulus between 100 kPa and 500 kPa. The dimensions of a typical solid tubular device has a diameter of 1.0 mm and a wall thickness of 0.1 mm. The device expands over 600% in diameter without breaking in an inflation balloon test. Scanning electron microphotographs of the device material typically show a range of pores between 0.1 and 5 microns. Endothelial cells, smooth muscle cells, and fibroblasts have been grown on top of the material surface, but they do not penetrate the material, demonstrating the separation barrier characteristic of the wall of the device. However, the tubular shape allows the transport or growth of cells in the axial direction of the tube, demonstrating the directional anisotropy of the device.

While the device will most commonly be of a tubular shape, other shapes are anticipated under this invention. For example, an elongated device with a polygonal or star-shape cross-section could provide similar function to prevent growth while allowing nutrient transport. An opening may be included in the side of the tubular shape to allow for flow to a branch. Similarly, a flat sheet could separate one layer of cells from another by having a pore size that excludes in-growth from one side. Further, a tubular shape with non-uniformly thick walls for additional mechanical strength is also anticipated.

Throughout this application, various publications are referenced. The disclosures of these publications in their entireties are hereby incorporated by reference into this application in order to more fully describe the state of the art to which this invention pertains.

The foregoing description has been presented for purposes of illustration and description. It is not intended to be exhaustive or to limit the invention to the precise examples or embodiments disclosed. Obvious modifications or variations are possible in light of the above teachings. The embodiment or embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly and legally entitled.

What is claimed is:

1. A medical implant made of an elastic, solid, biocompatible, and non-hemolytic device having a
thickness of less than about 5 mm, a modulus of elasticity between about 10 kPa and about 100 MPa,
and a pore size of less than about 10 microns that prevents growth and passage of cells through the implant device, while allowing water and nutrient transport across the implant device.

2. The medical implant claimed in claim 1 wherein the implant device has an ultimate stretch greater than 200 percent.

3. The medical implant claimed in claim 1 wherein the implant device is a synthetic organic polymer.

4. The medical implant claimed in claim 3 wherein the implant device is made from aqueous PVA.

5. The medical implant claimed in claim 1 wherein the implant device has an anisotropic cellular penetration through the boundaries of the implant.

6. The medical implant claimed in claim 5 wherein the implant device is tubular in shape.

7. The medical implant claimed in claim 6 wherein the tubular medical implant is open at one end.

8. The medical implant claimed in claim 6 wherein the tubular medical implant is open at both ends.

9. The medical implant claimed in claim 1 wherein the implant device is both radiolucent and clear.

10. The medical implant claimed in claim 1 wherein the implant device is hydrophilic and allows the inclusion of hydrophilic molecules.

11. A medical implant made of an elastic, solid, biocompatible, and non-hemolytic synthetic organic polymer device having a tubular shape,
a uniform thickness of less than about 5 mm, a modulus of elasticity between about 10 kPa and about 100 MPa, an ultimate stretch of greater than 200 percent,
and a pore size of less than about 10 microns that prevents growth and passage of cells through the surfaces of the device, while allowing anisotropic water and nutrient transport across the surfaces and ends of the device.

12. A method of making a semi-crystalline, solid, biocompatible and non-hemolytic implant device having a thickness of less than about 5 mm comprising:
mixing a solution of water and polyvinyl alcohol thereby forming an aqueous PVA mixture;
inserting a solid mandrel into said aqueous PVA mixture whereby the viscosity of said aqueous PVA mixture causes said aqueous PVA mixture to adhere to said mandrel;
spinning said mandrel within a non-aqueous environment or air in order to allow said aqueous PVA mixture to solidify at a uniform thickness about said mandrel;
freezing and thawing said aqueous PVA mixture at least once in order to create a semi-crystalline solid biocompatible implant device; and
removing said mandrel from said semi-crystalline solid biocompatible implant device.
